(19) United States
(12) Patent Application Publication
(10) Pub. No.: US 2026/0364531 A1
(43) Pub. Date: Nov. 19, 2026

(54) A CARBON DIOXIDE MANAGEMENT SYSTEM

(71) Applicant: Nobuyoshi MORIMOTO, Tokyo (JP)

(72) Inventor: Nobuyoshi MORIMOTO, Tokyo (JP)

(21) Appl. No.: 19/488,275

(22) PCT Filed: May 30, 2024
(86) PCT No.: PCT/IB2024/055265

§ 371 Date: Nov. 26, 2025

(30) Foreign Application Priority Data
May 30, 2023 (IN) 202311023766

(51) Int. Cl. B01D 53/84; B01D 53/14; B01D 53/18; B01D 53/34; B01D 53/62; B01D 53/75; B01D 53/94; C12P 3/00

(52) U.S. Cl. CPC B01D 53/84; B01D 53/1481; B01D 53/18; B01D 53/346; B01D 53/62; B01D 53/75; B01D 53/9454; C12P 3/00; B01D 2251/95; B01D 2257/104; B01D 2257/302; B01D 2257/404; B01D 2257/502; B01D 2257/504; B01D 2258/01

(57) ABSTRACT

The present invention provides a system and method to separate carbon dioxide from exhaust gases obtained from an engine, generators of a vessel and convert carbon dioxide into oxygen in a ballast tank of a vessel.

### FIELD OF INVENTION

The present invention generally relates to management of carbon dioxide (CO₂). More specifically, the present invention relates to a system and a method to manage carbon dioxide from exhaust gases obtained from an engine and generators of a vessel and convert the carbon dioxide into oxygen.

### BACKGROUND OF THE INVENTION

In shipping industry, heavy fuel oil is the most commonly used oil in engines of vessels to rotate propellers and provide required torque for moving the vessel forward. Heavy fuel oil is made from petroleum residue that remains after the higher-quality hydrocarbons have been extracted. Therefore, the heavy fuel oil is generally inexpensive. Combustion of heavy fuel oil in a vessel produces the highest amount of black carbon emissions.

Marine Diesel Oil, Marine Gas Oil and Bio fuels are generally used in vessels. These fuels are also made of hydrocarbons, and their combustion produces a large amount of pollution. These pollutants include oxides of Nitrogen (NO_{X}), oxides of Sulphur (SO_{X}), Carbon dioxide and soot particles.

The oxides of nitrogen (NO_{X}) combine with water and oxygen in the atmosphere and form highly corrosive nitrous and nitric acids. One such pollutant is nitrogen dioxide which is extremely poisonous and damages the lungs. Low-level ozone (O₃), which is a key pollutant and cause of smog, is produced at sea level when these gases interact with organic molecules. The oxides of sulphur, on the other hand, cause acid rains.

Moreover, carbon dioxide is a major greenhouse gas that contributes to global warming. It is the primary byproduct of hydrocarbons combustion in a vessel. Globally, great efforts have been made to reduce carbon emissions, in order to mitigate the effects of global warming.

In the shipping industry, reduction in carbon emissions is achieved gradually by reducing fuel consumption of the vessel. The reductions in fuel usage in ships are being achieved gradually through changes to the propulsion system, softer hull coatings, using air lubrication system and altered hull forms.

All of the above-mentioned methods used in the shipping industry are based on reducing fuel consumption. Thus far, no method is available to reduce carbon dioxide present in the exhaust gas and utilize carbon dioxide to convert it into the oxygen. Furthermore, there is a lot of interest in burning alternative fuels like green fuels, ammonia, LNG and others. However, these cannot solve the problem of decarbonization completely.

In view of the above limitations of the current technologies, there exists a need to develop a system and a method which can separate carbon dioxide from exhaust gases obtained from an engine of a vessel and convert the carbon dioxide into oxygen.

Thus, the above-described shortcomings of conventional approaches including devices/products and methods thereof, are merely intended to provide an overview of some of the problems of conventional approaches and are not intended to be exhaustive. Other problems with conventional approaches, and methods and their corresponding benefits of the various non-limiting embodiments described herein may become further apparent upon review of the following description.

### SUMMARY OF THE INVENTION

This summary is provided to introduce a selection of concepts in a simplified form that are further described below in the detailed description. This summary is not intended to identify key features or essential features of the claimed subject matter. Nor is this summary intended to be used to limit the claimed subject matter's scope.

Both the foregoing summary and the following detailed description provide examples and are only explanatory in nature. Accordingly, the foregoing summary and the following detailed description should not be considered to be restrictive. Further, features or variations may be provided in addition to those set forth herein. For example, embodiments may be directed to various feature combinations and sub-combinations described in the detailed description.

In view of the abovementioned problems in the associated art, it is an object of the present invention to provide system and method for converting carbon dioxide into oxygen within a vessel's ballast tank, leveraging the process of photosynthesis by phytoplankton.

It is another object of the present invention to tackle the challenge of vessel-based carbon dioxide emissions by creating a sustainable and eco-friendly closed-loop system. Phytoplankton are employed within a controlled environment to achieve efficient carbon dioxide (CO₂) conversion and oxygen (O₂) production.

It is yet another object of the present invention to provide a system for converting carbon dioxide to oxygen in a ballast tank of a vessel, the system comprising an engine or generator having an outlet which is fluidly connected to the ballast tank for receiving exhaust gases from the engine or generator. The ballast tank has phytoplankton from a water source and a light source. The system also has a circulation system for circulating the exhaust gases and phytoplankton within the ballast tank. The light source is adapted to promote photosynthesis by the phytoplankton, thereby converting carbon dioxide from the exhaust gases to oxygen.

It is still another object of the present invention to provide a gas separation unit operatively connected to the ballast tank for separating oxygen produced by the phytoplankton from the remaining gases.

It is another object of the present invention to provide a light source which is adapted to emit wavelengths suitable for photosynthesis.

It is another object of the present invention to provide the ballast tank equipped with absorbent material to absorb oxides of sulphur from the exhaust gases.

It is yet another object of the present invention to equip the first outlet with a three-way catalytic convertor to absorb oxides of nitrogen and carbon monoxide.

It is yet another object of the present invention that the three-way catalytic convertor to converts the oxides of nitrogen and carbon monoxide into carbon dioxide.

It is an object of the present invention to provide a method for converting carbon dioxide to oxygen in a ballast tank of a vessel wherein the method comprises introduction of exhaust gases from an engine or generator of the vessel into the ballast tank, introduction of phytoplankton from a water source into the ballast tank, exposition of the exhaust gases and phytoplankton within the ballast tank to light, and circulation of the exhaust gases and phytoplankton within the ballast tank. The light exposure promotes photosynthesis by the phytoplankton, thereby converting carbon dioxide from the exhaust gases to oxygen.

These and other objectives and embodiments of the invention will become more fully apparent when the following detailed description is read with the accompanying study details and examples. However, both the foregoing summary of the invention and the following detailed description of it represent one potential experiment and embodiment and are not restrictive of the invention or other alternate embodiments of the invention.

### DETAILED DESCRIPTION OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by a person skilled in the art should prevail.

Furthermore, it is important to note that, as used herein, “a” and “an” each generally denotes “at least one,” but does not exclude a plurality unless the contextual use dictates otherwise. When used herein to join a list of items, “or”, “/” denotes “at least one of the items,” but does not exclude a plurality of items of the list. Finally, when used herein to join a list of items, “and” denotes “all of the items of the list.”

Still further, the term “may” herein is used in a permissive sense (i.e. meaning having the potential to), rather than the mandatory sense, (i.e. meaning must).

Furthermore, the term “vessel” herein is used to represent a watercraft used for travel on water. It may be but not limited to a ship or a boat.

In one of the exemplary embodiments of the present invention, a system for separating carbon dioxide from exhaust gases obtained from an engine of a vessel and convert the carbon dioxide into oxygen is provided. Further, the present invention provides a method to separate carbon dioxide from exhaust gases obtained from an engine of a vessel and convert the carbon dioxide into oxygen.

The vessel as per the present invention includes but not limited to a cargo ship, a passenger ship, a defense ship, a research ship or a fishing ship.

Vessels generally comprise ballast tanks to provide hydrostatic stability to the vessel, reduce or control buoyancy, correct trim or list. The ballast tanks also provide an even load distribution along the hull of the ship to reduce structural hogging or sagging stress. During full load condition, i.e. when the vessel is fully loaded, the ballast tanks are kept empty or contain a minimum level of water, while during no load condition, i.e. when the vessel is not fully loaded, the ballast tanks are kept without water or filled with thin layer of water.

The ballast tank is generally completely dark due to the sealing structure to maintain the buoyancy to the ships. About ⅓rd of the ship displacement is utilized in the present invention for photosynthesis. Therefore, the invention may allow to reduce CO₂ emissions from engine or generator while sailing under laden and ballast condition.

The inlet for sea water in the ballast tank is capable of allowing an intake of phytoplankton. The phytoplankton is found in abundance, floating in the upper part of the water bodies, where sunlight penetrates the water. Phytoplankton may also be stored in small tank and the introduced into the ballast tank to convert CO₂ to oxygen. During no load condition, when the ballast tanks are filled with water, the phytoplankton may enter the ballast tanks or leave some air space by minimum ballast water to continue to deduct CO₂.

In a preferred embodiment of the present invention, the ballast tank comprises a light source in addition to an inlet for water. The light source may be an LED bulb, LED plant bulb, LED grow bulb or the like. The light source provides illumination to stimulate photosynthesis, and emits wavelengths within the Photosynthetically Active Radiation (PAR) range, specifically tailored to the chosen phytoplankton for optimal activity.

As per one of the exemplary embodiments of the present invention, the LEDs may be selected which emit specific wavelengths critical for plant growth, like Photosynthetically Active Radiation (PAR) in the 400-700 nm range. This reduces wasted energy on non-beneficial wavelengths. Further, cautious efforts are made to choose LEDs wherein high efficiency translates to less heat generation, minimizing the need for additional cooling and lowering energy consumption.

In another exemplary embodiment of the present invention, LEDs can be easily dimmed and programmed to create specific light cycles mimicking natural sunrise/sunset transitions.

In a preferred embodiment of the present invention, one end of an exhaust pipe is connected to the ballast tanks. Other end of the exhaust pipe may be connected to an engine and/or generators. The exhaust pipe may carry an exhaust gas from the engine to the ballast tanks. The exhaust gas may comprise oxides of Nitrogen (NO_{X}), oxides of sulphur (SO_{X}), Carbon Dioxide, Carbon Monoxide and soot particles. The vessel may be in full load condition or no-load conditions, the ballast tanks always have some space to accommodate the exhaust gas. Furthermore, the exhaust pipe pressure is lower than the inlet exhaust pressure from the engine/generators, allowing for smooth flow control. This is necessary to ensure that there is no back pressure to the engine and/or generator side.

In one of the exemplary embodiments of the present invention, emergency shutdown valves are provided for the continuous flow of engine and/or generator exhaust. The valves may be check-valves, non-return valves, back flow preventer valves or the like.

In one of the exemplary embodiments of the present invention, the exhaust pipe is constructed from robust materials resistant to high temperatures and potential corrosive elements present in the exhaust stream. Further, the pipe may be provided with heat insulation and potential condensation management for an increased efficiency.

In one of the exemplary embodiments of the present invention, the exhaust pipe comprises an absorbent material and a three-way catalytic convertor. The absorbent material may be configured to absorb oxides of Sulphur (SO_{X}). The three-way catalytic convertor may be configured to absorb oxides of Nitrogen (NO_{X}) and Carbon Monoxide and convert it into the Carbon Dioxide.

In one of the exemplary embodiments of the present invention, materials like activated alumina or zeolites capture sulfur oxides through adsorption. These materials have a porous structure that traps SO_{X} molecules on their internal surfaces.

In one of the exemplary embodiments of the present invention, the three-way catalytic converter uses platinum, rhodium, and palladium as catalysts. They work by facilitating reactions that convert oxides of nitrogen into N₂ and O₂, and CO into CO₂.

In one of the embodiments of the present invention, a robust circulation system is provided which continuously mixes the exhaust gases and phytoplankton culture within the ballast tank. Efficient mixing ensures even distribution of CO₂ throughout the tank volume. This maximizes gas exchange between the exhaust and the phytoplankton, promoting efficient CO₂ absorption.

The circulation system is designed to have an appropriate pump capacity, flow rate, and potential for creating turbulence that could disrupt the delicate phytoplankton culture.

In another embodiment of the present invention, alternative mixing methods such as baffles or induced air circulation, are used for better CO₂ absorption.

In a preferred embodiment of the present invention, the vessel has a gas separation unit. This unit separates oxygen (O₂) produced by the phytoplankton from the remaining gases in the exhaust mixture. The separation process may involve technologies like membrane separation or pressure swing adsorption.

In one of the exemplary embodiments of the present invention, an outlet pipe can be connected to the ballast tanks. The outlet pipe may be configured to carry out oxygen from the ballast tanks. As carbon dioxide has higher specific gravity in comparison of oxygen, carbon dioxide will stay at the level of sea water filled in the ballast tank and oxygen will float above carbon dioxide and escapes the ballast tank through the outlet pipe.

In an exemplary embodiment of the present invention, oxygen can be collected in dedicated storage tanks and utilized for various purposes onboard the vessel, such as supplementing the vessel's air supply or for various industrial applications.

In another exemplary embodiment of the present invention, the collected oxygen can be used to create bubbles along the periphery of the ship bottom, allowing the ship to glide over the water body at an increased speed.

In yet another exemplary embodiment of the present invention, the vessel may comprise an air compression device, wherein such air compression device may be connected to another end of the outlet pipe. The air compression device can receive oxygen from the outlet pipe, compress it and inject it above the sea water level to create air bubbles. Additionally, this compressor can control pressures in tanks by blowing unconverted CO₂ to the outside of ships.

In an exemplary embodiment of the present invention, the vessel may further comprise a nutrient tank to store nutrient which may be required by the phytoplankton. The nutrients stored in the nutrient tank may be nitrates, phosphates, sulfur or chlorella. The nutrient tank can thus be connected to the ballast tanks to provide nutrients to the phytoplankton.

In an exemplary embodiment of the present invention, the ballast tanks may comprise plurality of sensors to monitor the concentration of carbon dioxide inside the ballast tanks. The exhaust pipe may be continually or periodically adjusted to maintain the concentration of gases for efficient photosynthesis.

In another exemplary embodiment of the present invention, the ballast tanks may have inspection holes to allow a person to inspect the ballast tank. The inspection hole may be covered with a transparent cover creating an air tight seal. The transparent cover may be made out of glass or plastic.

In a preferred embodiment of the present invention, the method of conversion of carbon dioxide is disclosed, wherein carbon dioxide from the exhaust gas may reach the ballast tank. Further, the phytoplankton present in the ballast tank with the use of the light source, sea water present in the ballast tank and carbon dioxide start photosynthesis. By way of photosynthesis, the phytoplankton may convert the carbon dioxide and water into Oxygen and Glucose.

An exhaust pipe transports exhaust gases from the engine and generator to the ballast tank for organic conversion of carbon dioxide into oxygen. The ballast tank contains an LED plant bulb, an inlet for phytoplankton and sea water and is connected to the nutrient tank. The concentration of CO₂ is controlled with help of sensors, and phytoplankton transforms CO₂ to oxygen and glucose, and oxygen exists the ballast tank through the outlet pipe.

The photosynthesis process may occur 24/7 as long as an effective lighting is available in the ballast tanks. The key aspect is the separation of oxygen and carbon dioxide. As the carbon dioxide is heavier than oxygen, the ballast tanks are located close to carbon collection tanks in order to collect carbon dioxide separated from oxygen.

The foregoing descriptions of exemplary embodiments of the present disclosure have been presented for purposes of illustration and description. They are not intended to be exhaustive or to limit the disclosure to the precise forms disclosed, and obviously many modifications and variations are possible in light of the above teaching. The exemplary embodiment was chosen and described in order to best explain the principles of the disclosure and its practical application, to thereby enable others skilled in the art to best utilize the disclosure and various embodiments with various modifications as are suited to the particular use contemplated. It is understood that various omissions, substitutions of equivalents are contemplated as circumstance may suggest or render expedient, but is intended to cover the application or implementation without departing from the spirit or scope of the claims of the present disclosure.

1. A carbon dioxide management system for maritime vessels comprising:
a ballast tank configured to receive exhaust gases from the vessel, the ballast tank having
a water source within the ballast tank, the water source enriched with phytoplankton to facilitate carbon dioxide absorption from the received exhaust gases;
a light source positioned to illuminate the phytoplankton within the ballast tank;
a circulation system configured to promote the mixing of the exhaust gases with the phytoplankton-enhanced water to enhance carbon dioxide absorption.

2. The system as claimed in claim 1, wherein the light source promotes photosynthesis by the phytoplankton, and enables conversion of carbon dioxide from the exhaust gases to oxygen.

3. The system as claimed in claim 1, wherein the system further comprises a control unit operatively connected to the light source and the circulation system, configured to dynamically adjust the intensity and spectral output of the light source.

4. The system as claimed in claim 1, further comprising a gas separation unit operatively connected to the ballast tank for separating oxygen produced by the phytoplankton from the remaining gases.

5. The system as claimed in claim 1, wherein the light source is adapted to emit wavelengths suitable for photosynthesis.

6. The system as claimed in claim 1, wherein the ballast tank is equipped with absorbent material to absorb oxides of sulphide from the exhaust gases.

7. The system as claimed in claim 1, wherein the first outlet is equipped with three-way catalytic convertor to absorb oxides of nitrogen and carbon monoxide.

8. The system as claimed in claim 7, wherein the three-way catalytic convertor to converts the oxides of nitrogen and carbon monoxide into carbon dioxide.

9. A method for converting carbon dioxide to oxygen in a ballast tank of a vessel, comprising:
introducing exhaust gases from an engine or generator of the vessel into the ballast tank;
introducing phytoplankton from a water source into the ballast tank;
exposing the exhaust gases and phytoplankton within the ballast tank to light; and
circulating the exhaust gases and phytoplankton within the ballast tank,
wherein the light exposure promotes photosynthesis by the phytoplankton, thereby converting carbon dioxide from the exhaust gases to oxygen.

10. The method as claimed in claim 9, further comprising the step of separating the oxygen produced by the phytoplankton from the remaining gases in the ballast tank.
